(19) United States
(12) Patent Application Publication
(10) Pub. No.: US 2004/0009536 A1
(43) Pub. Date: Jan. 15, 2004

(54) System and method for predicting adme/tox characteristics of a compound

(76) Inventors: George Grass, Tahoe, CA (US); Glen D Leesman, Hamilton, CA (US); Daniel Norris, San Diego, CA (US); Patrick Sinko, Lebanon, NJ (US); Jehangir Athwal, San Diego, CA (US); Carleton Sage, Cardiff-by-the-Sea, CA (US); Troy Bremer, Dana Point, CA (US); Kevin Holme, San Diego, CA (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/332,997

(22) PCT Filed: Jul. 30, 2001
(86) PCT No.: PCT/US01/23763

(51) Int. Cl. G01N 33/53; G01N 33/567; G06G 7/48; G06G 7/58; G06F 19/00; G01N 33/48; G01N 33/50; G01N 31/00

(52) U.S. Cl. 435/7.2; 702/19; 702/22; 703/11; 703/12

(57) ABSTRACT

A method for developing a predictive model of a chemical compound property. The method includes obtaining at least one descriptor from structural data for each of a plurality of compounds. At least one chemical compound property is obtained for each of the plurality of compounds. The predictive model is developed by mapping the at least one descriptor to the chemical compound property. The chemical compound property may be an ADME property. The ADME property may be absorption. The chemical compound property may also be an toxicity property.

This application claims the benefit of U.S. Provisional Application Nos. 60/221,548 filed Jul. 28, 2000, entitled PHARMACOKINETIC-BASED DRUG DESIGN TOOL AND METHOD; and 60/267,435 filed Feb. 9, 2001 entitled SYSTEM AND METHOD FOR PREDICTING ADME CHARACTERISTICS OF A COMPOUND BASED ON ITS STRUCTURE.

### BACKGROUND OF THE INVENTION

1. Field of the Invention

The present invention relates to systems and methods for predicting the characteristics of a chemical compound. In particular, the present invention is related to pharmacokinetic systems and methods for predicting the Absorption, Distribution, Metabolism, Excretion and/or Toxicological (ADME/TOX) characteristics or properties of a chemical compound based on structural modeling of the chemical compound and mathematical analysis.

2. Description of the Prior Art

Pharmacodynamics refers to the study of fundamental or molecular interactions between drug and body constituents, which through a subsequent series of events results in a pharmacological response. For most drugs, the magnitude of a pharmacological effect depends on the time-dependent concentration of drug at the site of action (e.g., target receptor-ligand/drug interaction). Factors that influence rates of delivery and disappearance of drug to or from the site of action over time include its ADME properties. The study of factors that influence how drug concentration varies with time is the subject of pharmacokinetics. Additionally, the toxicological properties of a drug should also be considered. These properties taken together represent the ADME/TOX properties of a compound.

In nearly all cases, the site of drug action is located on the other side of a membrane from the site of drug administration. For example, an orally administered drug must be absorbed through a series of physiological barriers at some point or points along the gastrointestinal (GI) tract. Once the drug is absorbed, and thus passes a membrane barrier of the GI tract, it is transported through the portal vein to the liver and then eventually into systemic circulation (i.e., blood and lymph) for delivery to other body parts and tissues by blood flow. Thus, how well a drug crosses membranes is of key importance in assessing the rate and extent of absorption and distribution of the drug throughout different body compartments and tissues. In essence, if an otherwise highly potent drug is administered extravascularly (e.g., oral) but is poorly absorbed (e.g., GI tract), a majority of the drug will be excreted or eliminated and thus cannot be distributed to the site of action.

The ADME/TOX properties of a candidate drug (chemical compound) are usually determined through conventional laboratory testing (in vitro or in vivo) combined with mathematical modeling. For instance, pharmacokinetic data analysis may be based on empirical observations after administering a known dose of drug to an animal and fitting of the data collected from the animal (e.g., from its liver cells) by either descriptive equations or mathematical (compartmental) models. Time-concentration data from a subject that has been given a particular dose of a drug may be collected followed by plotting the data points on a logarithmic graph of drug concentration versus time to generate one type of concentration-time curve. A mathematical equation is used to model what might happen to the drug as it is transported through a human body. Classical one, two and three compartment models used in pharmacokinetics require in vivo blood data to describe concentration-time effects related to the drug decay process, i.e., blood data is relied on to provide values for equation parameters. For instance, while a model may work to describe the decay process for one drug, it is likely to work poorly for others unless blood profile data and associated rate process limitations are generated for each drug in question. Thus, current models are very poor for predicting the in vivo fate of diverse drug sets in the absence of blood data and the like derived from animal and/or human testing (Lipinski et al. 1997. Advanced Drug Delivery Reviews. 23, 3-25; Palm et al. 1997. Pharm. Res. 14(5) 568-571). For this reason, animal testing is still very much used to predict the ADME/TOX properties of chemical compounds. However, several studies have shown that in general, such types of testing in animal models are poor surrogates for performance in humans (W. K. Sietsema, Int. J. Clin. Pharmacol, Therapy, and Toxicol., 27:179-211 (1989)). Furthermore, conventional laboratory testing and animal testing is very costly and time consuming.

Thus, there is a need for new and improved systems and methods for predicting the ADME/TOX characteristics of chemical compounds that can eliminate or reduce the need for animal testing as well as all other types of physical experimental testing. These new systems will also improve the correlation to the true needed endpoint, which, in most cases is man.

### SUMMARY OF THE INVENTION

The present invention solves the aforementioned problems by providing new and improved systems and methods of predicting the ADME/TOX properties of candidate drugs (chemical compounds). Such systems and methods may use empirical statistical pattern recognition approaches to take known chemical structures and characteristics (e.g., ADME/TOX) of all compounds for which data has been generated (e.g., data is available from various labs, is published, etc.) and to relate the structures and their characteristics to experimental data in such a way to accurately predict the characteristics of a new proposed structure (compound).

According to an embodiment of the present invention, provided is a system for predicting the target data of a compound in a mammalian (actual descriptions are human related) body comprising a database facility and a processor facility. The database facility is configured to store input data. The processor facility is configured to allow the entry of input data relating to a new proposed chemical compound including structural data, to perform an analysis of the chemical compound by mapping the data entered to produce predicted target data for the chemical compound based on the analysis.

According to another embodiment of the present invention, provided is a method for creating or developing a model to be used for evaluating the ADME/TOX characteristics of a proposed compound. The method comprises the following steps:

(a) selecting training compounds based on the characteristics to be predicted of the proposed compounds (for which a complete set of input and target data exists)

(b) selecting descriptors applicable to the characteristic to be predicted based on an analysis of the training compounds selected in step (a), such as via a genetic algorithm or other appropriate mathematical analysis

(c) mapping the training set obtained in (b) to the target data resulting in a model which could predict the target data of a proposed compound.

Compounds should be selected for their applicability for the problem to be solved, for example, such as for Caco-2 effective permeability (Caco-2 cells possess many of the properties of the small intestine; as such, these cells represent a useful and well-accepted tool for studying the absorption and/or secretion of drugs/chemicals across the intestinal mucosa). Accordingly, drugs may be selected as compounds to be analyzed because of their proven permeability or absorption properties. Other compounds may similarly be selected and added to the data set. Once compounds have been analyzed for descriptors, they may be tested by conventional means (e.g., lab testing, etc.) to determine various characteristics to be predicted by the system above (e.g., CaCo-2 permeability). Once all data has been analyzed and collected, they are loaded into the database for use in predicting the ADME/TOX properties of proposed compounds.

In other embodiments, the method may include:

(a) receiving at least one proposed compound (e.g., the molecular structure, etc.) via a user input means (e.g., from a file, input via a form, etc.),

(b) selecting training compounds from the database facility based on the characteristics to be predicted of the proposed compounds (for which a complete set of input and target data exists)

(c) selecting the most meaningful descriptors applicable to the characteristic to be predicted based on an analysis of the training compounds selected in step (b), such as via a genetic algorithm or other appropriate mathematical analysis

(d) creating validation data subsets of the training data based upon the distribution of descriptors and target characteristics of compounds selected in (b/c)

(e) mapping the training set obtained in (d) to the target data resulting in a model which could predict the target data of a proposed compound.

(f) modifying (for example: boosting, bootstrap aggregation (bagging)), and other model enhancement methods, etc.) one or more models produced in (e) based upon performance on validation sets obtained in (d) to form a composite model

(g) combining (via boosting, committee machines etc,) a set of two or more models produced in (e or f) based upon performance on validation sets obtained in (d) to form a composite model

(h) running the model determined in either step (e), (f) or (g) using the required input data (the identity of the subset of input data itself. was determined in step (c)) to predict the required target data

According to another embodiment of the present invention, provided is a system for predicting the chemical properties of at least one proposed compound comprising: a database facility configured to store and to serve input data relating to the characteristics of training compounds (descriptor(s) (for example, structure and experimental data)) as well as target data (for example, chemical properties of selected compounds) for the training compounds; and a processor facility coupled to the database facility and configured to predict the characteristics of a proposed compound by:

(a) selecting training compounds from the database facility based on the characteristics to be predicted of the proposed compounds (for which a complete set of input and target data exists)

(b) selecting descriptors applicable to the characteristic to be predicted based on an analysis of the training compounds selected in step (a), such as via a genetic algorithm or other appropriate mathematical analysis

(c) mapping the training set obtained in (b) to the target data resulting in a model which could predict the target data of a proposed compound.

According to another embodiment of the present invention, provided is a system for predicting the chemical properties of at least one proposed compound comprising: a database facility configured to store and to serve input data relating to the characteristics of the proposed compound (descriptor(s) (for example, structure and experimental data)); and a processor facility coupled to the database facility and configured to predict the characteristics of a proposed compound by:

(a) receiving at least one proposed compound (e.g., the molecular structure, etc.) via a user input means (e.g., from a file, input via a form, etc.),

(b) running the model using the appropriate input data to predict the required target data

According to another embodiment of the present invention, provided is a system for predicting the chemical properties of at least one proposed compound comprising: a database facility configured to store and to serve input data relating to the characteristics of training compounds (descriptor(s) (for example, structure and experimental data)) as well as target data (for example, chemical properties of selected compounds) for the training compounds; and a processor facility coupled to the database facility and configured to predict the characteristics of a proposed compound by:

(a) receiving at least one proposed compound (e.g., the molecular structure, etc.) via a user input means (e.g., from a file, input via a form, etc.);

(b) selecting training compounds from the database facility based on the characteristics to be predicted of the proposed compounds (for which a complete set of input and target data exists);

(c) selecting the most meaningful descriptors applicable to the characteristic to be predicted based on an analysis of the training compounds selected in step (b), such as via a genetic algorithm or other appropriate mathematical analysis;

(d) creating validation data subsets of the training data based upon the distribution of descriptors and target characteristics of compounds selected in (b/c);

(e) mapping the training set obtained in (d) to the target data resulting in a model which could predict the target data of a proposed compound;

(f) modifying (for example: boosting, bootstrap aggregation (bagging)), and other model enhancement methods, etc.) one or more models produced in (e) based upon performance on validation sets obtained in (d) to form a composite model;

(g) combining (via boosting, committee machines etc,) a set of two or more models produced in (e or f) based upon performance on validation sets obtained in (d) to form a composite model; and

(h) running the model determined in either step (e), (f) or (g) using the required input data (the identity of the subset of input data itself was determined in step (c)) to predict the required target data.

Analysis used to select the most meaningful subset of input data (step (c)) for predicting target data may be performed via feature selection methods such as forwards or backwards selection and may include regression/classification methods. Such analyses should consider model bias and overtraining.

The preceding analyses may include various data compression techniques.

A particular model may be biased if the training data is poorly distributed (e.g. the distribution has sharp peaks, regions between nodes that are devoid of data, etc). Accordingly, compounds may be selected and tested to improve the distribution and enhance the model's ability to generalize. Furthermore, the input's and target's distributions along with the proposed compound's descriptors and characteristic values are used to calculate a confidence metric.

The methods and applications described herein have been limited in scope to the ADME/Tox area. It should be understood that these methods are generally applicable to any research area where chemical structure is to be correlated with some experimental or otherwise determined property. Examples would be QSAR modeling for molecule potency and/or specificity, toxicological profiles of molecules, physicochemical properties of molecules (solubility, melting point), etc.

### BRIEF DESCRIPTION OF THE DRAWINGS

FIG. 1. is a block diagram of a system for predicting the ADME/Tox properties of a candidate drug;

FIG. 2 is a flow chart of the method for developing a model that will predict the ADME/Tox properties of a candidate drug; and for predicting the ADME/Tox properties of a candidate drug.

FIGS. 3-45 are individual showings of particular points pertinent and important to the present invention and illustrate specific examples of an embodiment of the invention aimed at predicting human ADME data.

### DESCRIPTION OF SPECIFIC EMBODIMENTS

1. Definitions

The following bolded terms are used throughout this document with the following associated meanings:

Absorption: Transfer of a compound across a physiological barrier as a function of time and initial concentration. Amount or concentration of the compound on the external and/or internal side of the barrier is a function of transfer rate and extent, and may range from zero to unity.

Affine Regression: Linearly combining input data to approximate output data. This is essentially a linear regression that does not require the regression to go through zero.

Bioavailability: Fraction of an administered dose of a compound that reaches the sampling site and/or site of action. May range from zero to unity. Can be assessed as a function of time.

Boosting: A general method which attempts to increase the accuracy of a learning algorithm.

Compound: Chemical entity. Could be a drug, a gene, etc.

Computer Readable Medium: Medium for storing, retrieving and/or manipulating information using a computer. Includes optical, digital, magnetic mediums and the like; examples include portable computer diskette, CD-ROMs, hard drive on computer etc. Includes remote access mediums; examples include internet or intranet systems. Permits temporary or permanent data storage, access and manipulation.

Cross Validation: Used to estimate the generalization error. This method is based on resampling the data set, using randomly (or otherwise chosen) samples of the training set as test sets.

Data: Experimentally collected and/or predicted variables. May include dependent and independent variables.

Input Data: Data which is used as an input in the training or execution of a model. Could be either experimentally determined or calculated.

Target Data: Data for which a model is generated. Could be either experimentally determined or predicted.

Test Data: Experimentally determined data.

Descriptor: An element of the input data.

Committee Machine: A model that is comprised of a number of submodels such that the knowledge acquired by the submodels is fused to provide a superior answer to any of the independent submodels.

Regression/Classification: Methods for mapping the input data to the target data. Regression refers to the methods applicable to forming a continuous prediction of the target data, while classification (or in general pattern recognition) refers the methods applicable to separating the target data into groups or classes. The specific methods for performing the regression or classification include where appropriate: Affine or Linear Regressions, Kernel based methods, Artificial Neural Networks, Finite State Machines using appropriate methods to interpret probability distributions such as Maximum A Posteriori, Nearest Neighbor Methods, Decision Trees, Fisher's Discriminate Analysis.

Mapping: The process of relating the input data space to the target data space, which is accomplished by regression/classification and produces a model that predicts or classifies the target data.

Feature Selection Methods: The method of selecting desirable descriptors from the input data to enable the prediction or classification of the target data. This is typically accomplished by forward selection, backward selection, branch and bound selection, genetic algorithmic selection, or evolutionary selection.

ADME: Properties of absorption, distribution, metabolism, and excretion and encompasses other measures related to absorption, distribution, metabolism, and excretion. For example, heptocyte turnover or Caco-2 effective permeability.

Dissolution: Process by which a compound becomes dissolved in a solvent.

Fisher's Discriminate Analysis: A linear method which reduces the input data dimension by appropriately weighting the descriptors in order to best aid the linear separation and thus classification of target data.

Genetic Algorithms: Based upon the natural selection mechanism. A population of models undergo mutations and only those which perform the best contribute to the subsequent population of models.

Input/Output System: Provides a user interface between the user and a computer system.

Kernel Representations: Variations of classical linear techniques employing a Mercer's Kernel or variations to incorporate specifically defined classes of nonlinearity. These include Fisher's Discriminate Analysis and principal component analysis. Kernel Representations as used by the present invention are described in the article, “Fisher Discriminate Analysis with Kernels,” Sebastian Mika, Gunnar Ratsch, Jason Weston, Bernhard Scholkopf, and Klaus-Robert Muller, GMD FIRST, Rudower Chaussee 5, 12489 Berlin, Germany, ©IEEE 1999 (0-7803-5673-X/99), and in the article, “GA-based Kernel Optimization for Pattern Recognition: Theory for EHW Application,” Moritoshi Yasunaga, Taro Nakamura, lkuo Yoshihara, and Jung Kim, IEEE© 2000 (0-7803-6375-2/00), which are both hereby incorporated herein by reference.

Metabolism: Conversion of a compound (the parent compound) into one or more different chemical entities (metabolites).

Artificial neural networks: A parallel and distributed system made up of the interconnection of simple processing units. Artificial neural networks as used in the present invention are described in detail in the book entitled, “Neural networks, A Comprehensive Foundation,” Second Edition, Simon Haykin, McMaster University, Hamilton, Ontario, Canada, published by Prentice Hall ©1999, which is hereby incorporated herein by reference.

Permeability: Ability of a barrier to permit passage of a substance or the ability of a substance to pass through a barrier. Refers to the concentration-dependent or concentration-independent rate of transport (flux), and collectively reflects the effects of characteristics such as molecular size, charge, partition coefficient and stability of a compound on transport. Permeability is substance and/or barrier specific.

Physiologic Pharmacokinetic Model: Mathematical model describing movement and disposition of a compound in the body or an anatomical part of the body based on pharmacokinetics and physiology.

Principal Component Analysis: A type of non-directed data compression which uses a linear combination of features to produce a lower dimension representation of the data. An example of principal component analysis as applicable to use in the present invention is described in the article, “Nonlinear Component Analysis as a Kernel Eigenvalue Problem,” Bernhard Scholkopt, Neural Computation, Vol. 10, Issue 5, pp. 1299-1319, 1998, MIT Press., and is hereby incorporated herein by reference.

Simulation Engine: Computer-implemented instrument that simulates behavior of a system using an approximate mathematical model of the system. Combines mathematical model with user input variables to simulate or predict how the system behaves. May include system control components such as control statements (e.g., logic components and discrete objects).

Solubility: Property of being soluble; relative capability of being dissolved.

Support Vector Machines: Method which regresses/classifies by projecting input data into a higher dimensional space. Examples of Support Vector machines and methods as applicable to the present invention are described in the article, “Support Vector Methods in Learning and Feature Extraction,” Berhard Scholkopf, Alex Smola, Klaus-Robert Muller, Chris Burges, Vladimir Vapnik, Special issue with selected papers of ACNN'98, Australian Journal of Intelligent Information Processing Systems, 5 (1), 3-9), and in the article, “Distinctive Feature Detection using Support Vector Machines,” Partha Niyogi, chris Burges, and Padma Ramesh, Bell Labs, Lucent Technologies, USA, IEEE ©1999 (0-7803-5041-3/99), which are both hereby incorporated herein by reference.

2. Preferred Embodiments

There are roughly four major properties involved in human pharmacokinetics: Absorption, Distribution, Metabolism, and Elimination (ADME). For example, when a drug is taken into the body orally, the first thing that has to happen is it has to get absorbed into the body in GI tract. From there, the drug travels to the liver via the portal vein where it is either metabolized or not. After the drug passes through the liver it is distributed throughout the body. Once the drug is distributed throughout the body, it is transported to the kidney to get eliminated. The effectiveness of a drug (a chemical compound) is directly related to the way a body will absorb, distribute, metabolize and eliminate the compound. In addition to the ADME properties of a compound, the toxicological effects of the compound should also be considered. The present invention is directed to systems and methods for predicting various characteristics (ADME/Tox characteristics) related to the way a body will absorb, distribute, metabolize, eliminate, and respond to potential toxic effects of a compound based on the compound's chemical structure and/or associated experimental data.

The molecular structure of a proposed compound may be input as a 2-dimensional (2D) connection table, which is essentially a two-dimensional graph of how the atoms of a compound are arranged (the structures may actually be 3-dimensional (3D), but may be represented as 2D via well known methods). Alternatively, the structure may be input as a 3D structure. Either 2D or 3D structural representations are desirable inputs for models using structure to predict ADME/Tox characteristics.

There are really three fundamental properties of the molecule that decide whether or not it's a drug: the first is whether or not it actually interacts with a particular molecular target in the body (in most cases, some kind of protein); the second is whether or not the body can absorb, metabolize, distribute and eliminate the compound adequately, and third, whether or not the compound elicits a toxic response.

The present invention provides systems and methods for predicting the ADME/Tox properties (e.g., Caco-2 effective permeability or Caco-2 Peff), of a proposed compound through statistical analysis of compound data. By using the present invention, it is therefore possible to significantly reduce the need for expensive and time consuming testing, such as animal testing, because the ADME/Tox characteristics of an untested compound is predicted with a high level of accuracy.

The first section of the present invention employs mathematical analyses of a diverse compilation of training data (chemical compound data including conventional experimental results, chemical descriptor analysis, etc.) to determine what data relates to the ADME/Tox property to be predicted. Once the type or types of data that are applicable to the ADME/Tox property (descriptors) are determined, mathematical analyses of the selected training data to obtain the selected ADME/Tox characteristic for each training data compound are performed in order to create a model. The model can then be used to predict a proposed compound's ADME/Tox property by inputting the same type of data for the proposed compound into the model. Running the model with the proposed compound's descriptors produces the predicted ADME/Tox characteristic.

Models are only as good as the input assay and test data, and therefore, a key to producing highly accurate predictions is the use of well-defined standard operating procedures for generating data as well as insuring that the data has a good distribution. Therefore, the present invention provides a method for collecting and compiling a diverse training data set to be used to mathematically predict the ADME/Tox characteristics of a proposed chemical compound.

The input data is collected and/or calculated for a variety of chemical compounds preferably representing currently prescribed drugs as well as failed drugs and potential new drugs (this is a continual process, since as more data is collected, the resulting models will have improved performance). Assay data may be collected from well established sources or derived by conventional means. For instance, in vitro assays characterizing permeability and transport mechanisms may include in vitro cell-based diffusion experiments and immobilized membrane assays, as well as in situ perfusion assays, intestinal ring assays, incubation assays in rodents, rabbits, dogs, non-human primates and the like, assays of brush border membrane vesicles, and averted intestinal sacs or tissue section assays. In vivo assay data typically are conducted in animal models such as mouse, rat, rabbit, hamster, dog, and monkey to characterize bioavailability of a compound of interest, including distribution, metabolism, elimination and toxicity. For high-throughput screening, cell culture-based in vitro assays or biochemical assays from isolated cell components or recombinantly expressed components are preferred. For high-resolution screening and validation, tissue-based in vitro and/or mammal-based in vivo data are preferred.

Cell culture models are preferred for high-throughput screening, as they allow experiments to be conducted with relatively small amounts of a test sample while maximizing surface area and can be utilized to perform large numbers of experiments on multiple samples simultaneously. Cell models or biochemical assays also require fewer experiments since there is no animal to animal variability. An array of different cell lines also can be used to systematically collect complementary input data related to a series of transport barriers (passive paracellular, active paracellular, carrier-mediated influx, carrier-mediated efflux) and metabolic barriers (protease, esterase, cytochrome P450, conjugation enzymes).

Cells and tissue preparations employed in the assays can be obtained from repositories, or from any eukaryote, such as rabbit, mouse, rat, dog, cat, monkey, bovine, ovine, porcine, equine, humans and the like. A tissue sample can be derived from any region of the body, taking into consideration ethical issues. The tissue sample can then be adapted or attached to various support devices depending on the intended assay. Alternatively, cells can be cultivated from tissue. This generally involves obtaining a biopsy sample from a target tissue followed by culturing of cells from the biopsy. Cells and tissue also may be derived from sources that have been genetically manipulated, such as by recombinant DNA techniques, that express a desired protein or combination of proteins relevant to a given screening assay. Artificially engineered tissues also can be employed, such as those made using artificial scaffolds/matrices and tissue growth regulators to direct three-dimensional growth and development of cells used to inoculate the scaffolds/matrices. It will be understood that ideally any known test results could be added to a test data set in order to adjust the model or to provide a new property to solve towards.

The drugs (compounds) selected should be as diverse in character as possible. Therefore, the compounds may be analyzed and defined in chemical space. Chemical space can be represented as an N-base coordinate system in which to plot compounds and may be used to show the diversity of a sample of compounds. The axes of N-base coordinate system may be selected from all or some of the input data. Drugs may be eliminated from a particular training data set (the training data may be grouped to solve for a particular ADME/Tox property) if it is determined that they bias the training data set.

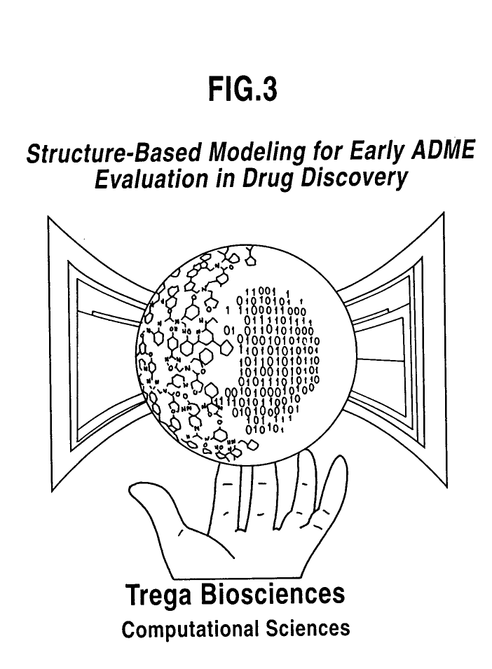

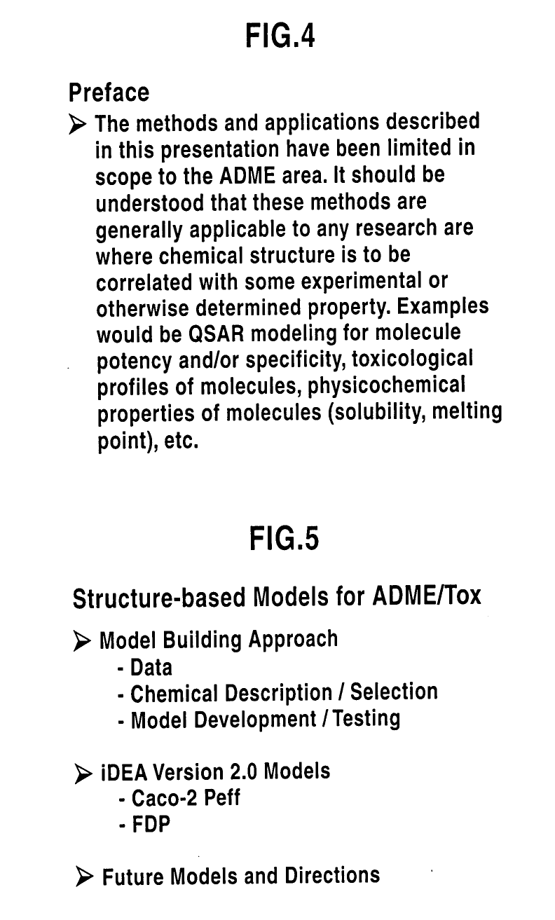

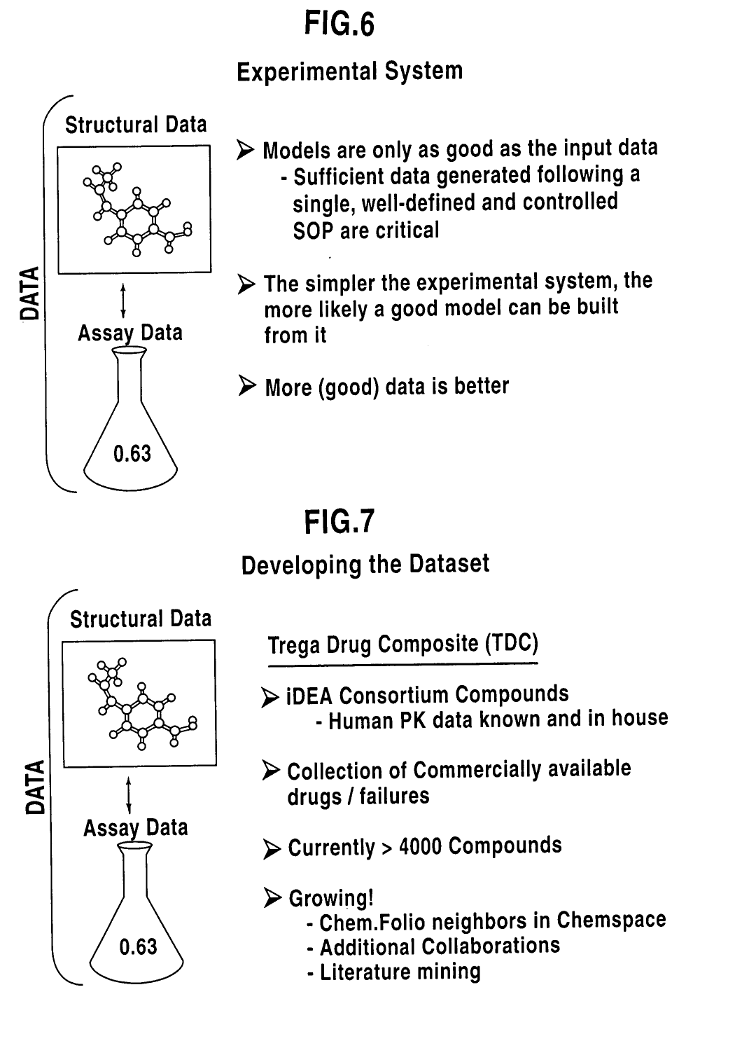

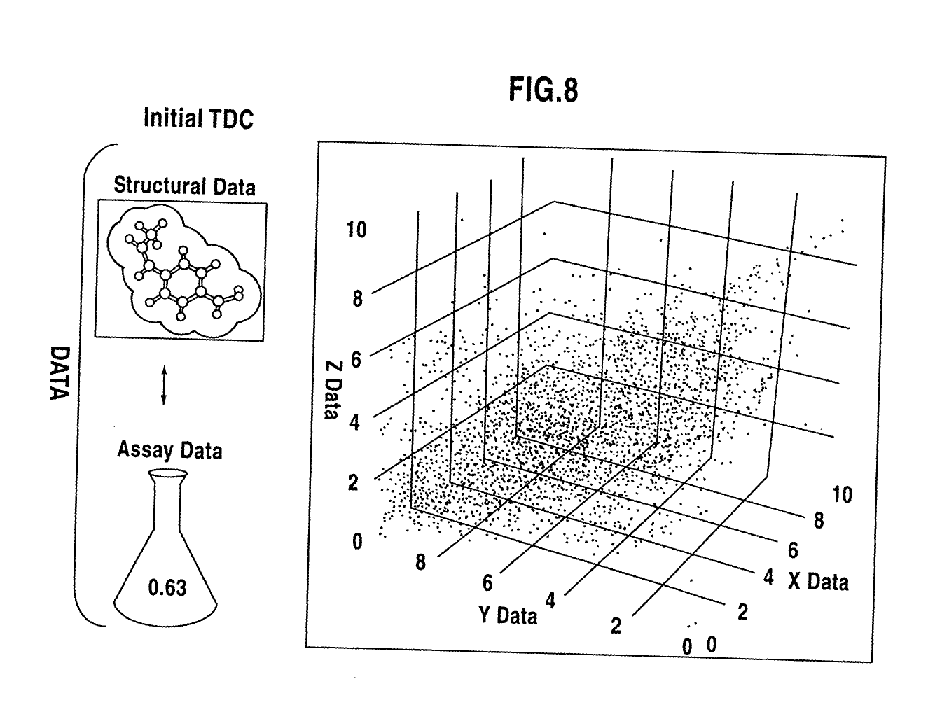

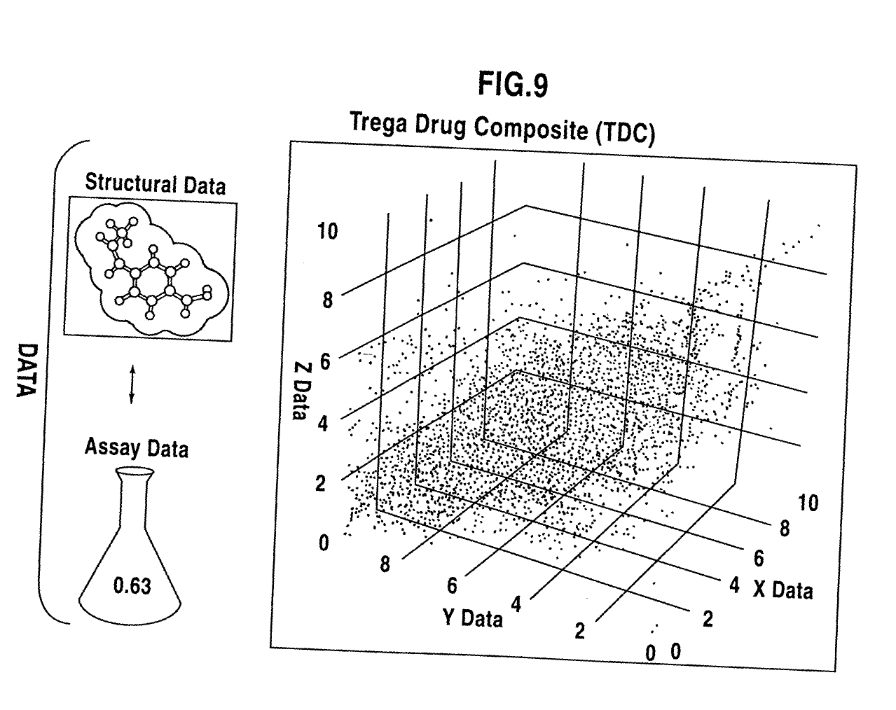

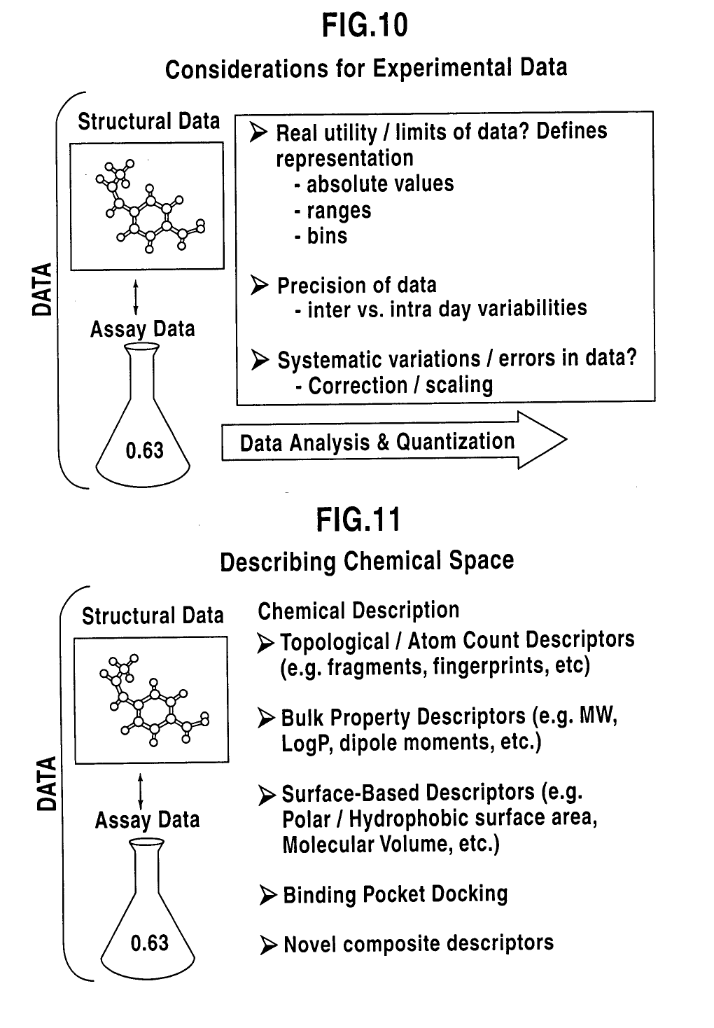

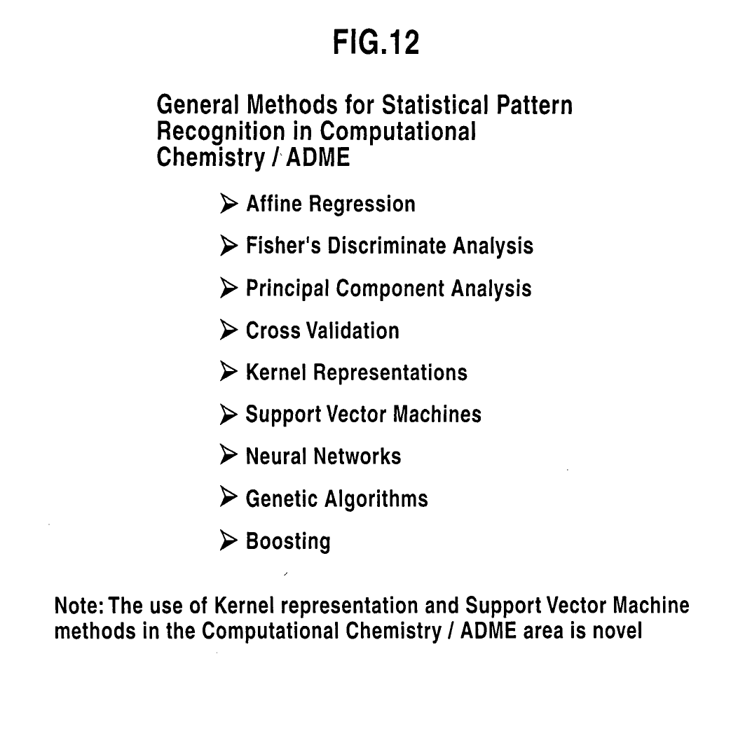

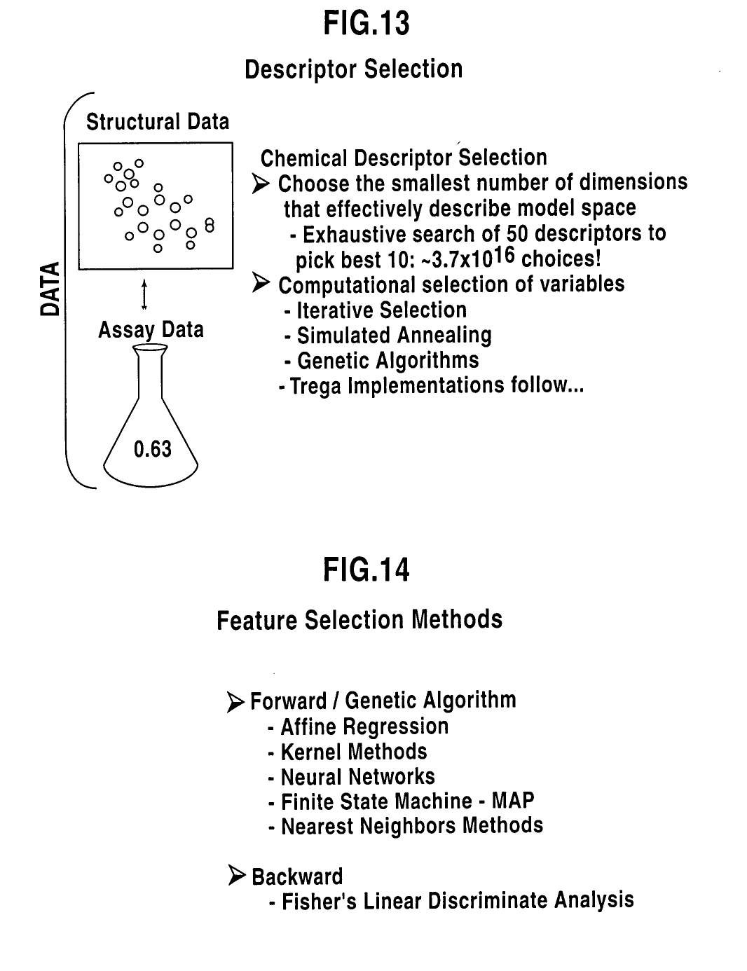

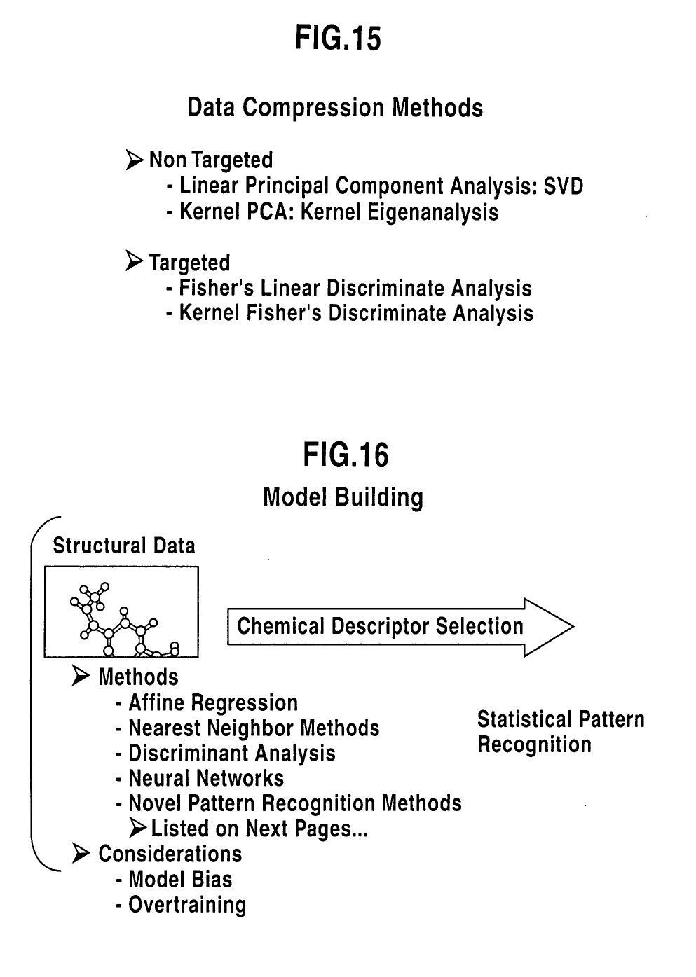

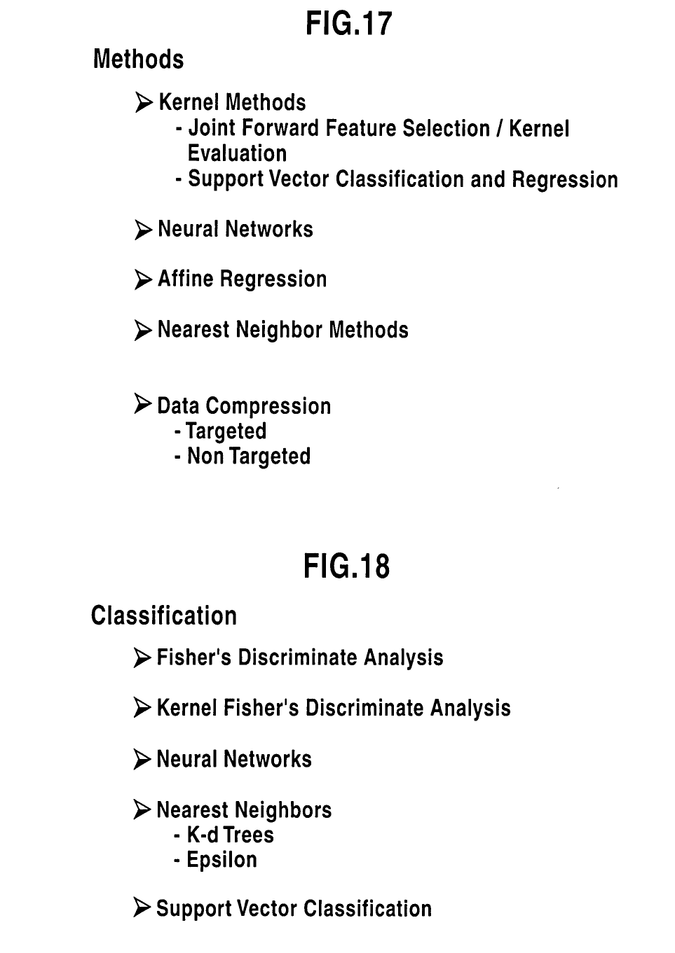

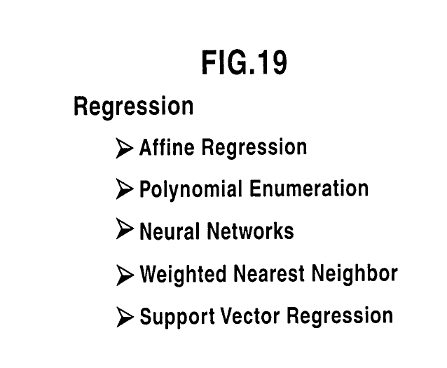

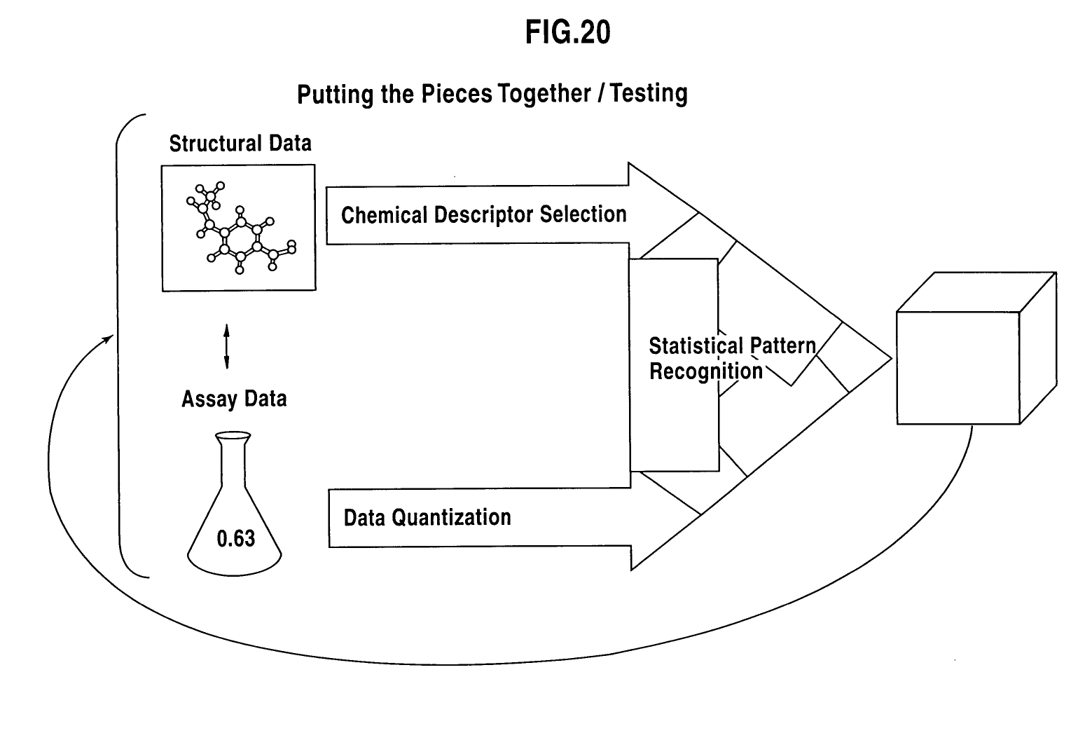

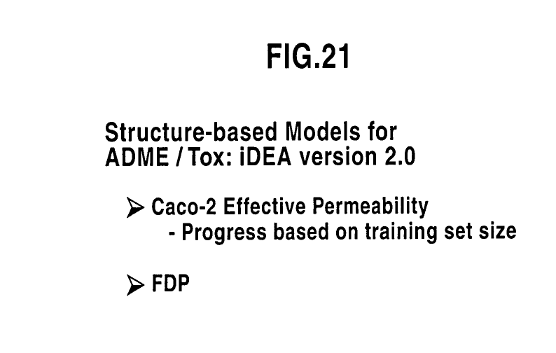

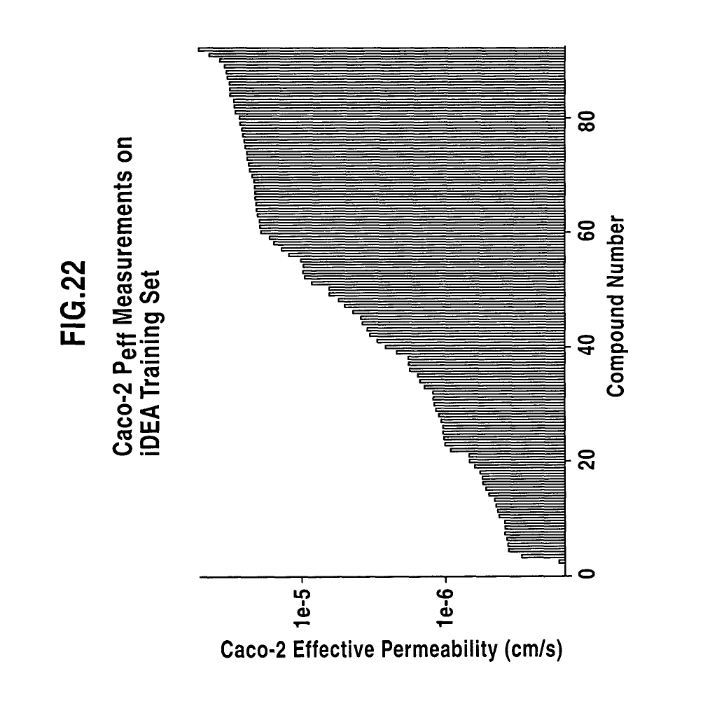

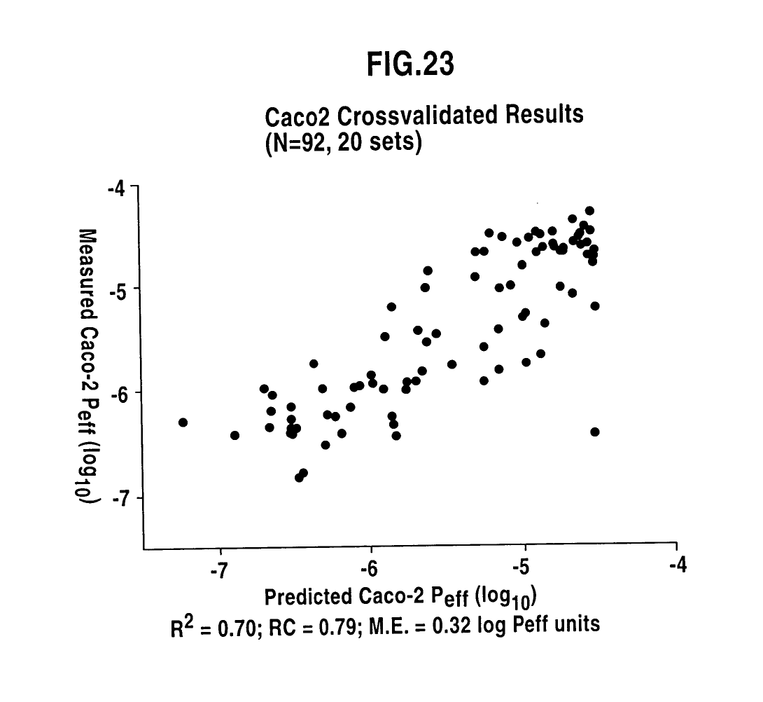

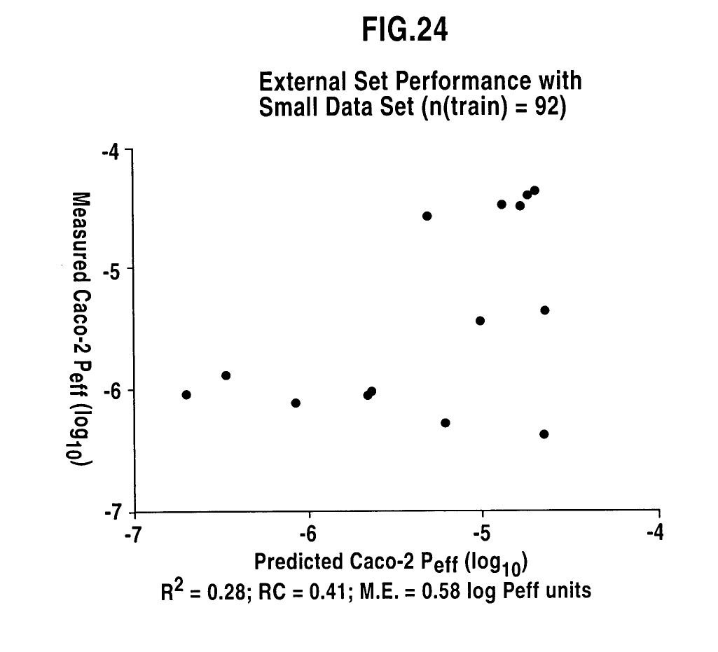

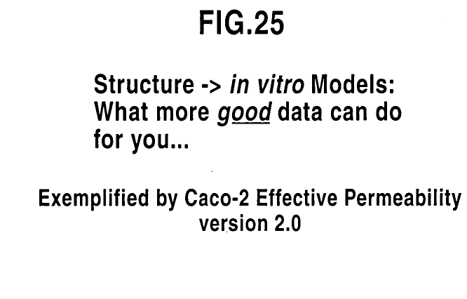

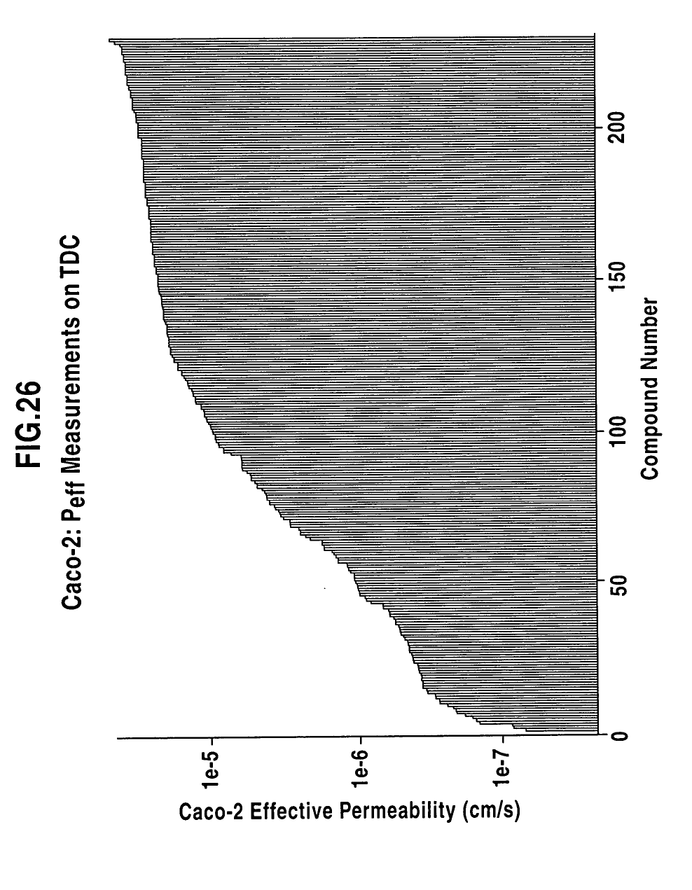

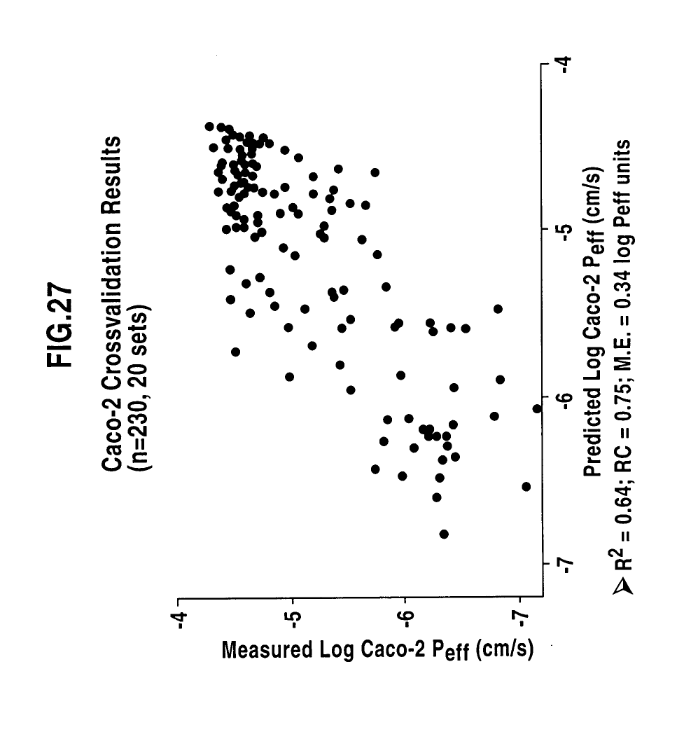

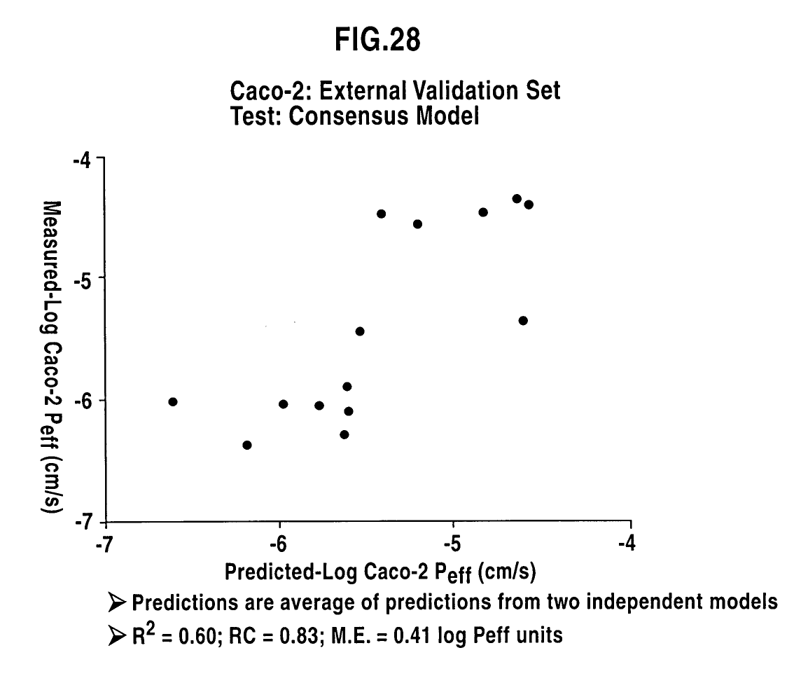

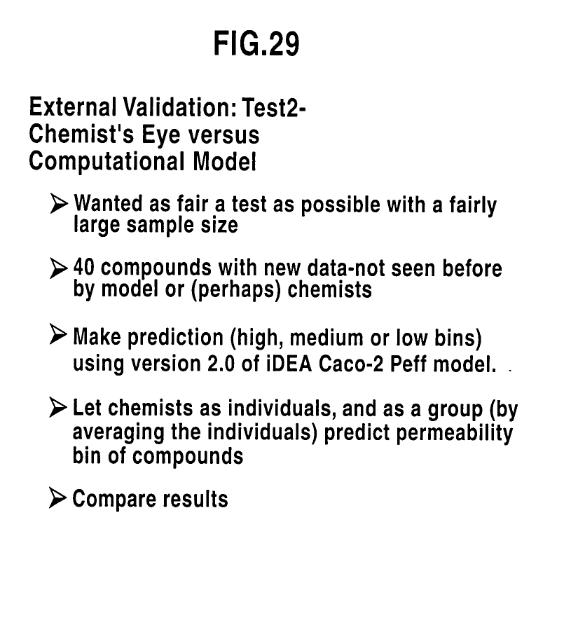

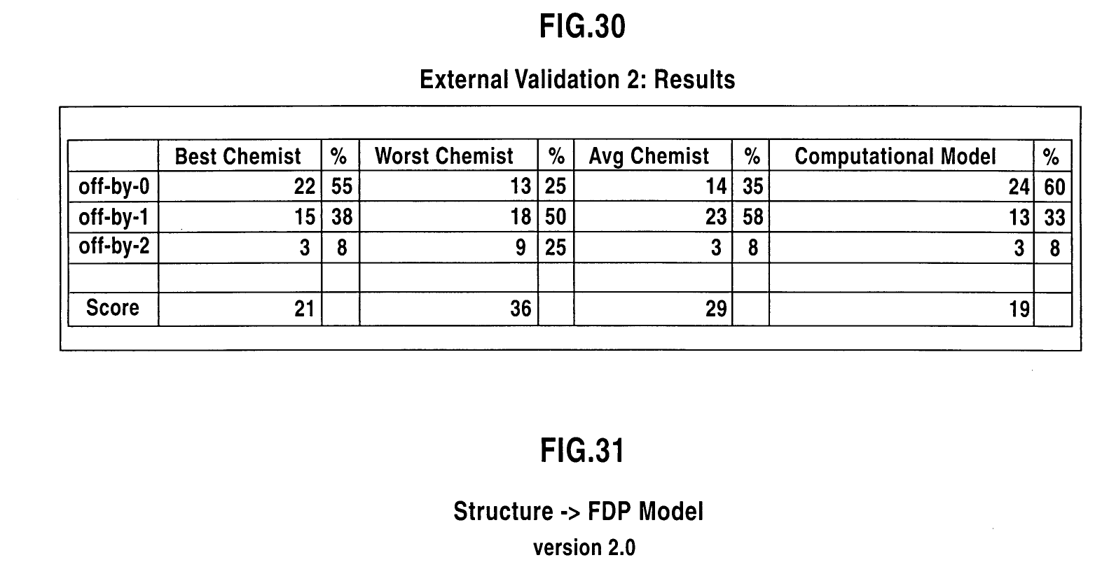

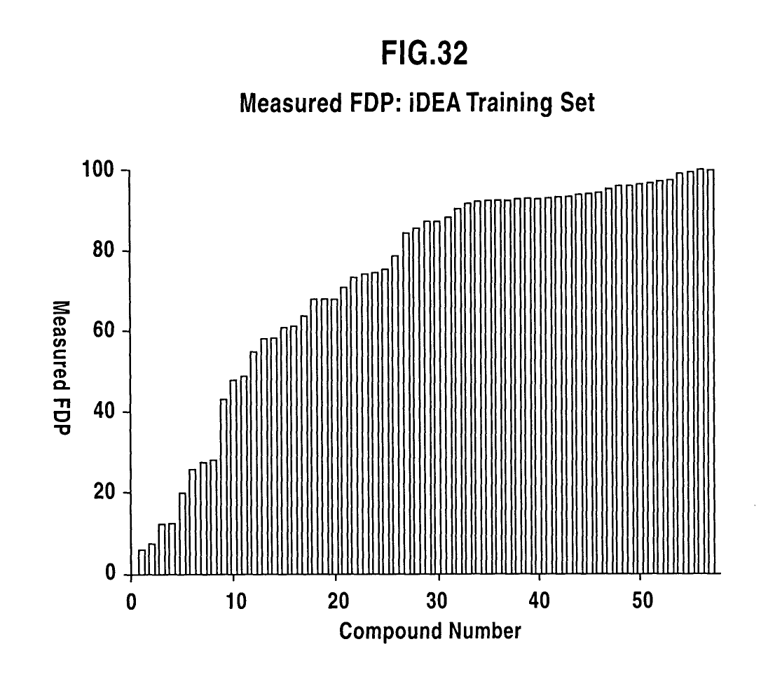

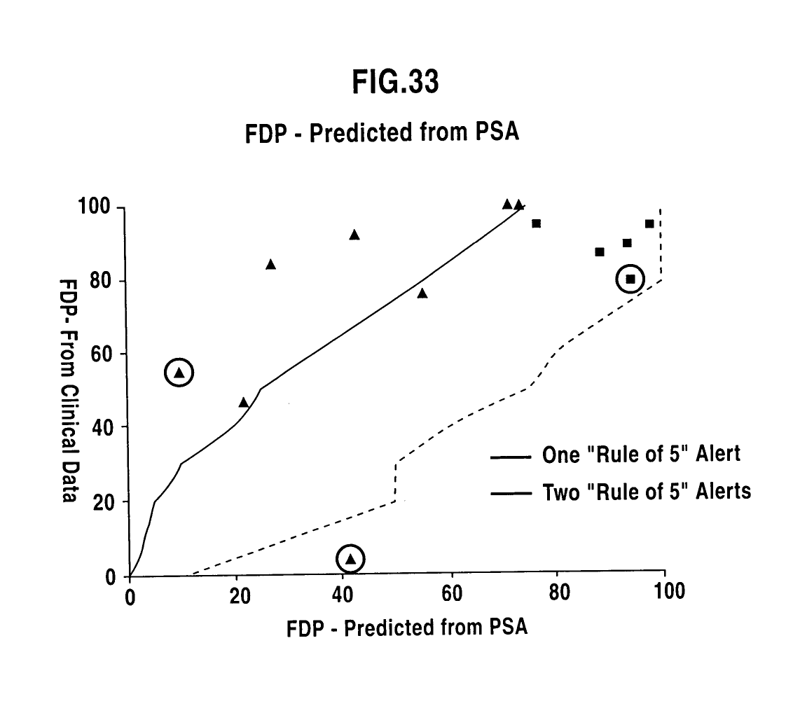

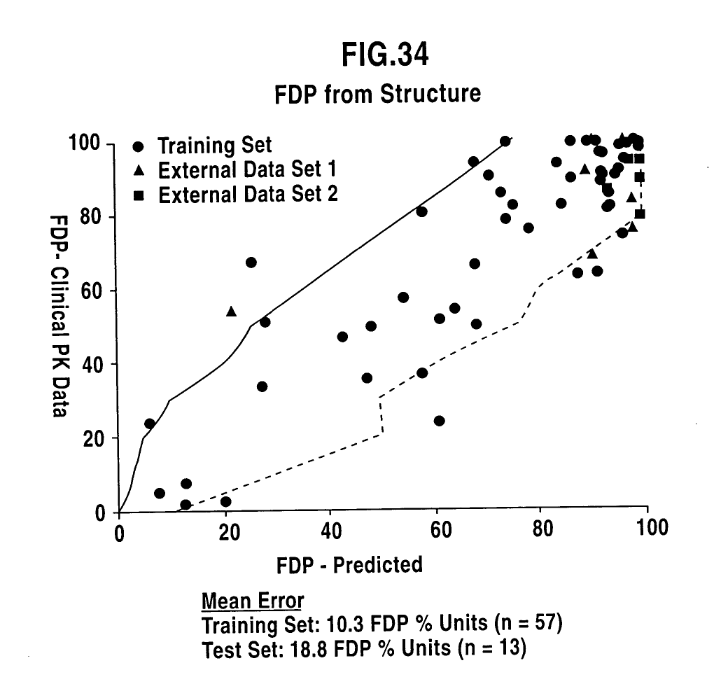

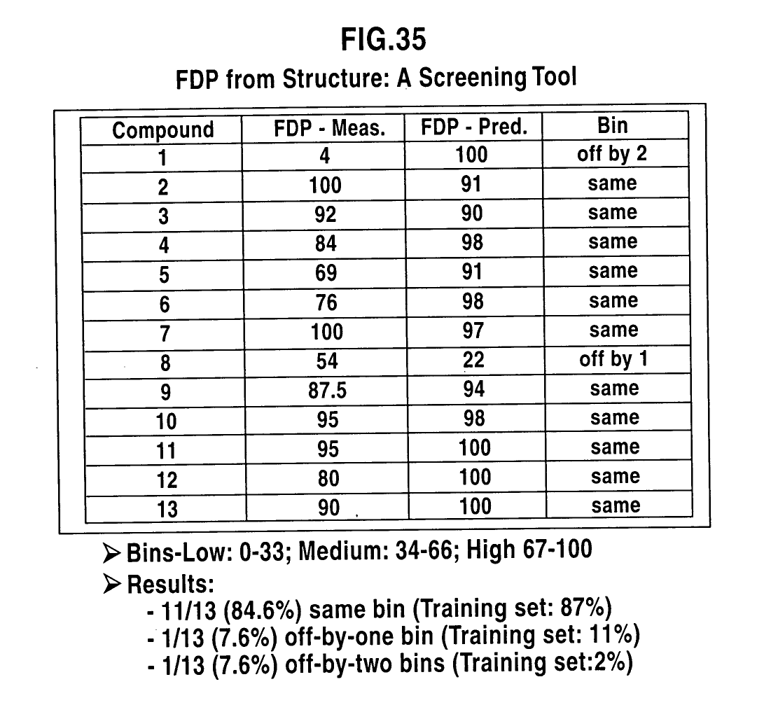

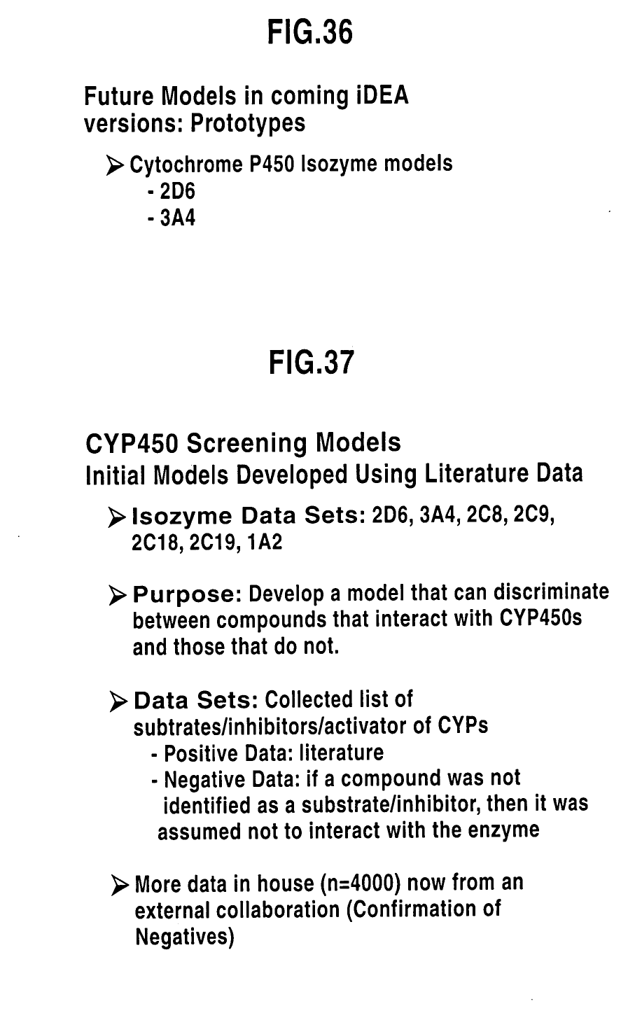

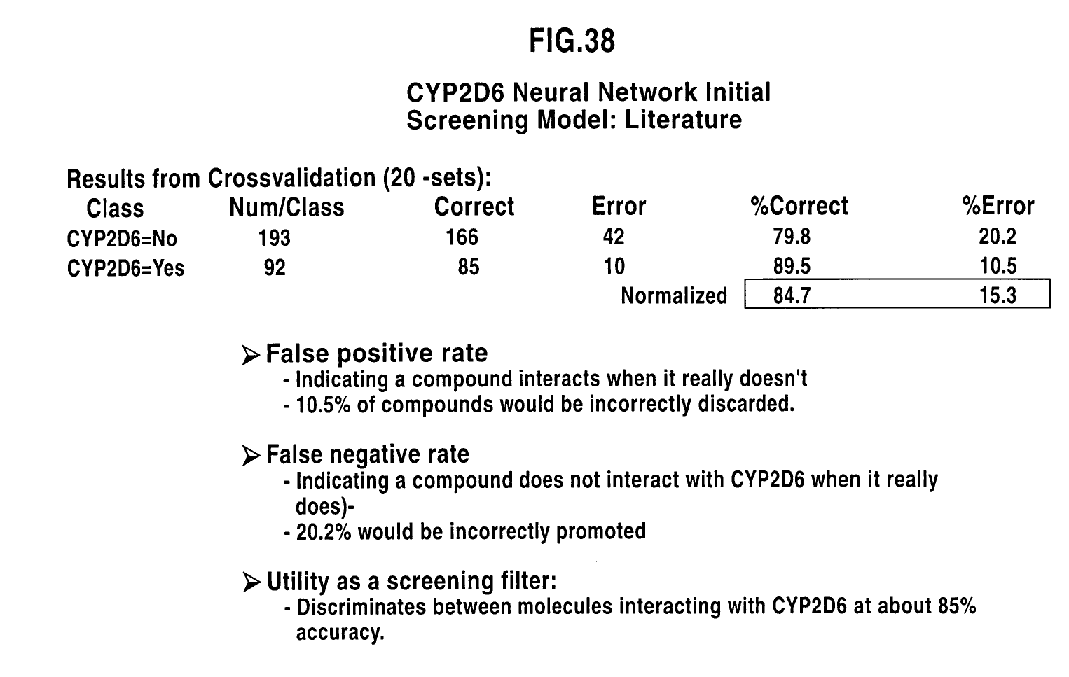

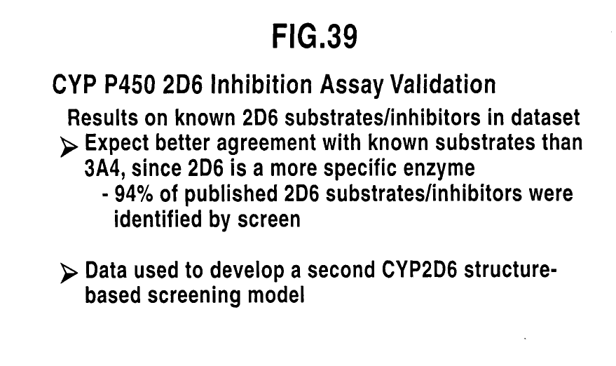

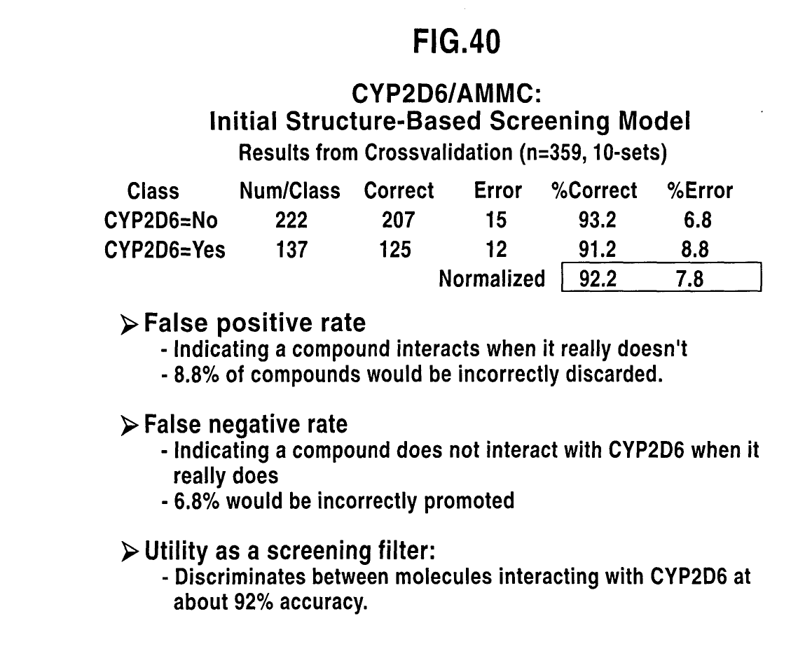

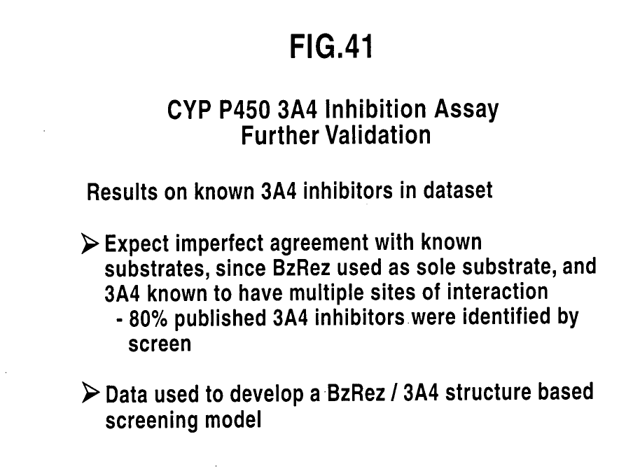

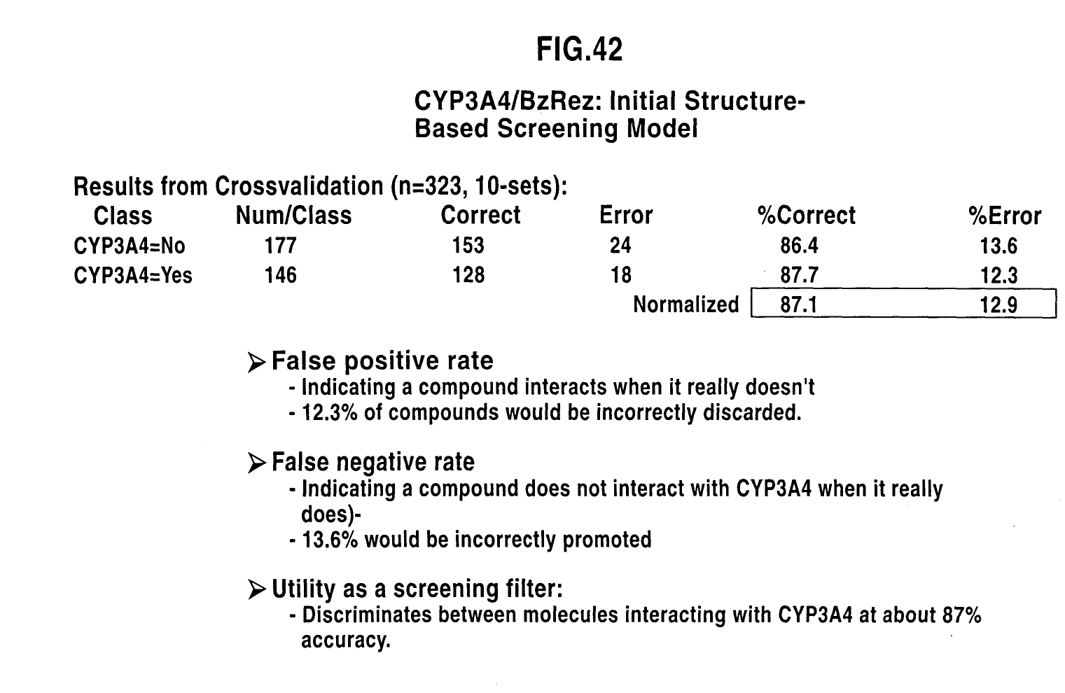

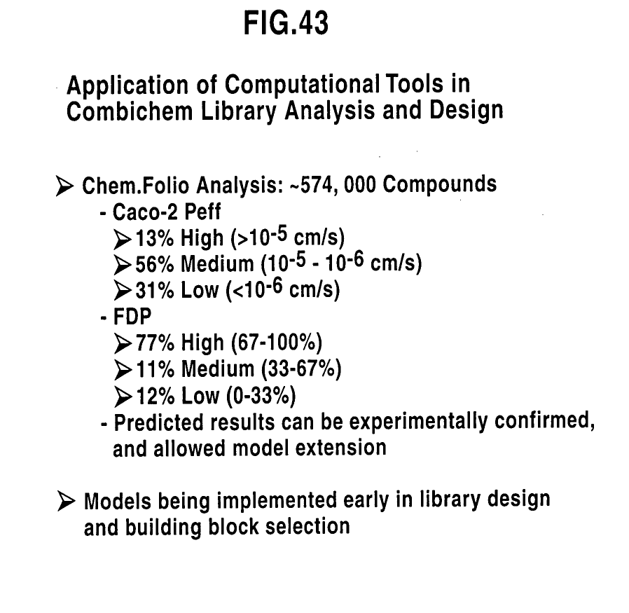

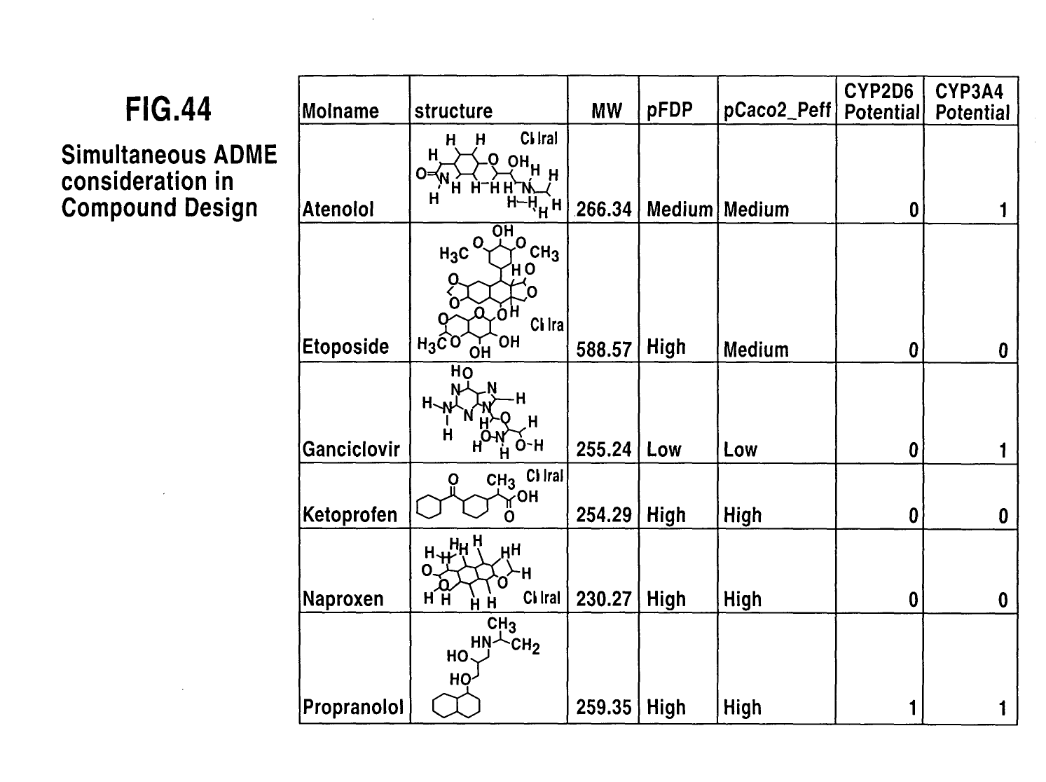

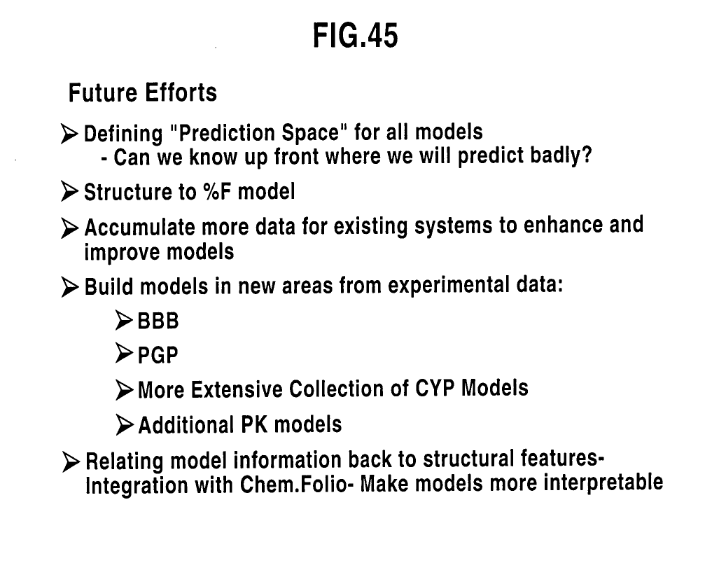

In the present invention, a collection of drugs have been plotted in a six-base chemical space (see FIG. 3). The axes of the six-base are physicochemical descriptors that were selected so that the best separation of known drugs is maintained. Data is also selected from combinatorial libraries of chemicals which are near neighbors for each of the drugs creating an extended data set. The compounds are ideally each tested for various ADME/Tox characteristics or properties to be predicted, however it is not necessary to test every compound for actual results.

There are many considerations for the experimental data. Each data set of experimental data is analyzed to decide how it is going to be used in model building. For example, is it appropriate to use a certain data set to predict absolute values of compounds or is there too much error in the data set? If there is not enough data in a data set to cover a particular range (either coverage in the data space, representation in the data space, or certainty in the data space) it is possible to put the data into bins, such as 0 to 20, 21 to 40, 41 to 60, 61 to 80, 81 to 100. Alternatively, the data may require scaling correction to account for systematic variations in the data. One having ordinary skill in the art will readily understand the grouping of experimental data, scaling and systematic variations used to adjust a data set.

Next, a tool is used to calculate additional data by analyzing each compound and describing the compound with chemical descriptors. Chemical descriptors are well known in the art of modeling compounds, and may be determined by analyzing a 2D or 3D structure of a compound.

Finally, all the training data (input and target data) collected or created is compiled and preferably maintained in a relational database or other known means for making the data easily accessible and available to be manipulated and analyzed in accordance with the present invention.

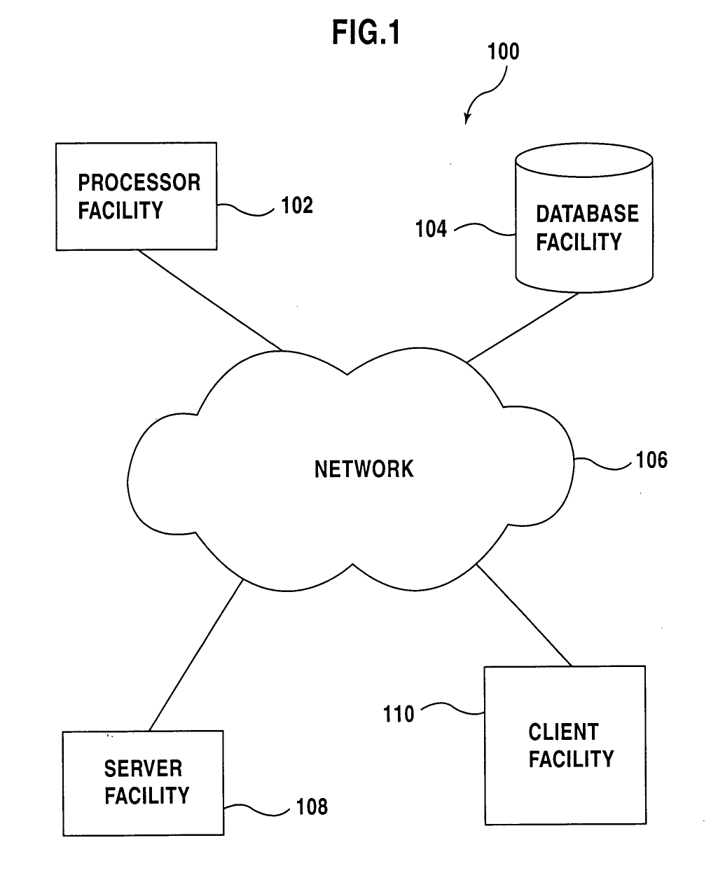

The present invention is now described with reference to FIG. 1. In particular, system 100 includes a processor facility 102 and a data facility 104 coupled to a network 106. The processor facility 102 may be a conventional computer, such as a PC, configured to access database facility 104 and to execute analytical software in accordance with the present invention. Database facility 104 may be a conventional database server running a database engine, such as SQLSERVER® or ORACLE 8i® and is configured to maintain and to serve data, such as the test data described above. The data may be stored and maintained by any means such as in a relational dataspace or an objected oriented dataspace.

The present invention includes analytical tools which may be executed on processor facility 102. The analytical tools may be in the form of software that is loaded locally on processor facility 102 or may be served via a server 108 (e.g., an HTML form, JAVA program, etc. served on a web server), which optionally may be included. Accordingly, a client facility 110 may be connected to the network 106, which may include parts of the Internet and World Wide Web (WWW), or local area networks (LANS). The client facility 110 could be a web browser or other terminal configured to access and run the analytical tools remotely or to download the analytical tools (e.g., via HTML, IIOP, etc.) via network 106 and run them locally.

The configuration of system 100 is merely exemplary and is not meant to limit the present invention. It will be appreciated that the present invention may take many forms and configurations. For example, the present invention may be implemented via a software solution including a database and forms configured to run on a stand-alone PC, or may alternatively be a combination of software and firmware, and may be implemented in a client-server, stand-alone or web configuration.

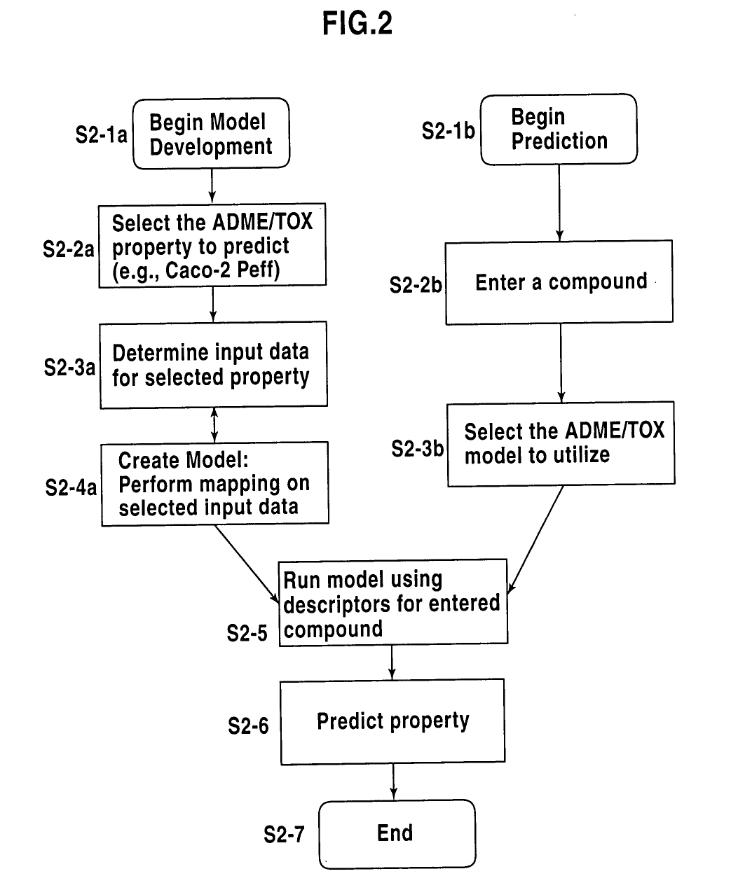

The operational aspects of the present invention are now described with reference to the flow chart in FIG. 2. The flow chart represents two independent starting pathways which meet at step S2-5, a model development pathway, and a model execution or prediction pathway, these two initial pathways will be described independently.

Model Development Pathway (S2-1a->S2-5)

The model development pathway begins in step S2-1a and immediately proceeds to step S2-2a. At step S2-2a, the ADME/Tox property to be predicted is selected. For example, it may be desired to predict the Caco-2 Peff of the compound, or the FDP (fraction of the dose administered that is absorbed at the portal vein). The system might allow for the selection to be from a table, radio group, pop-list, or by any known means. Also at step S2-2a, a set of training compounds appropriate for developing the selected ADME/Tox property model is entered into the system. Many compound descriptors may be entered or calculated, such as molecular weight, structure, specific gravity, etc.

Next, at step S2-3a, a group of meaningful input data is selected based on the property to be predicted or a related performance metric using feature selection methods. For example, a genetic algorithm coupled with a regression/classification method, such as a neural network, may be used to build many models predicting the Caco-2 Peff of a compound. Features are then selected from the resulting models with the objective of choosing the smallest number of dimensions that effectively describe the model space. One should keep in mind when performing the analyses to select a number of descriptors which avoids biased and non-predictive models (e.g., overtraining).

Once the descriptors have been selected, a model is created at step S2-4a by using regression/classification methods to map the input data to the ADME/Tox property to be predicted. The modeling effort may involve Affine Regressions, Nearest Neighbor Methods, Discriminate Analysis, Support Vector Machines, Artificial neural networks, Data Compression techniques (targeted and non-targeted), Genetic Algorithms, and Boosting. In addition, a method for calculating a confidence metric is created by analyzing information related to the model such as the distributions and values of the input and target data and the methods involved in building the model.

It should be noted that instead of predicting continuous values for a specific ADME/Tox property, the present invention may be used to classify a particular compound (e.g., can it be absorbed, is it toxic, etc.). A compound is classified by the same method predicting a specific ADME/Tox property, except that the analyses performed may vary slightly, and the classifications are performed to solve for a “yes/no” or “high, medium, low” binning type solution (e.g., 1-bit).

The model resulting from step S2-4a is used in step S2-5 to predict new proposed compounds in the model execution pathway.

Model Execution Pathway (S2-1b->S2-7)

Once the model has been created/developed, then the model may be used to predict the ADME/Tox property of the proposed compound. The model execution pathway begins at step S2-1b, and proceeds directly to S2-2b where at least one proposed compound may be entered.

Next, at step S2-3b, the property to be predicted is selected. For example, it may be desired to predict the Caco-2 Peff of the compound, or the FDP. The system might allow for the selection to be from a table, radio group, pop-list, or by any known means.

Next, at step S2-5, the descriptors for the proposed compound (identified in step S2-3a)) are input into the model created in step S2-4a. The model is run and a result (e.g., a Caco-2 Peff or FDP prediction) is produced in step S2-6. As described above, a measure of confidence in the result may also be produced.

Processing terminates at step S2-7.

It should be readily apparent to one having ordinary skill in the art that the preceding method may be implemented via numerous configurations. For example, the preceding method and analysis therein may be implemented via a C++ program coupled to a data warehouse, or alternatively may be implemented via a combination of program components and databases.

Heretofore, only highly trained pharmacokinetic experts were capable of determining and therefore, estimating a compound's ADME/TOX. Moreover, such estimations usually included very time consuming and costly experimentation. The present invention now provides a less expensive and time consuming, and potentially more accurate means for predicting the ADME characteristics of proposed drugs, and therefore, by using the present invention, many individuals and entities will now be able to more affordably screen compounds for their applicability as drugs before any animal testing or other lab testing is necessary.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the invention.

We claim:

1. A method for developing a model to predict a chemical compound property, the method comprising:
obtaining at least one descriptor from structural data for each of a plurality of compounds;
obtaining at least one descriptor from experimental or predicted data for each of a plurality of compounds;
obtaining at least one chemical compound property for each of the plurality of compounds; and
developing the model by mapping the descriptors to the chemical compound property.

2. The method of claim 1, wherein the chemical property is an ADME property.

3. The method of claim 2, wherein the ADME property is absorption.

4. The method of claim 2, wherein the ADME property is Caco-2 Effective Permeability.

5. The method of claim 1, wherein the chemical property is a toxicity property.

6. The method of claims 1-5 wherein obtaining at least one descriptor comprises selecting the descriptors applicable to the characteristic to be predicted based on an analysis of the plurality of compounds.

7. The system of claim 6 wherein the analysis used to select the descriptors for predicting the characteristic is selected from at least one of the following: Affine Regressions, Kernel Methods, Artificial neural networks, Finite State Machines—Maximum A Posteriori, Nearest Neighbor Methods, Fisher's Linear Discriminate Analysis, or other regression/classification methods.

8. The system of claim 6 further comprising:
performing a chemical space analysis of the plurality of compounds;
if the chemical space analysis indicates that the plurality of compounds selected should be modified to improve diversity of the chemical space, then modifying the plurality of compounds by addition or deletion of a compound to improve the diversity of the chemical space covered by the plurality of compounds.

9. A system for predicting an ADME/Tox of a compound in a mammalian body, the system comprising:
a database facility, the database facility configured to store and to provide structural and experimental or predicted data; and
a processor facility, the processor facility configured to allow the entry of data relating to a new proposed chemical compound including structural data and experimental or predicted data, to perform an analysis of the chemical compound by mapping the data entered to produce a predicted ADME/Tox property of the chemical compound based on the analysis.

10. A method for compiling chemical compound data to be used for evaluating the characteristics of a proposed compound, the method comprising:
selecting a plurality of compounds;
obtaining a descriptor analysis for each of the plurality of compounds;
obtaining test results related to the characteristics being evaluated; and
loading the descriptor analysis and the test results into a database used to predict the characteristics of proposed compounds.

11. The method of claim 10 further comprising:
performing a chemical space analysis of the plurality of compounds;
if the chemical space analysis indicates that the plurality of compounds selected should be modified to improve diversity of the chemical space, then modifying the plurality of compounds by addition or deletion of a compound to improve the diversity of the chemical space covered by the plurality of compounds.

12. A system for predicting the chemical properties of a proposed compound comprising:
a database facility configured to store and to serve data relating to the characteristics of selected compound, including structure data, descriptor data, and test data; and
a processor facility coupled to the database facility and configured to predict the characteristics of a proposed compound by:
(a) receiving at least one proposed compound via a user input means;
(b) selecting training compounds from the database facility based on the characteristics to be predicted of the proposed compounds;
(c) selecting the most meaningful descriptors applicable to the characteristic to be predicted based on an analysis of the training compounds selected in step (b);
(d) creating validation data subsets of the training data based upon the distribution of descriptors and target characteristics of compounds selected in (b/c);
(e) mapping the training set obtained in (d) to the target data resulting in a model which could predict the target data of a proposed compound;
(f) modifying (for example: boosting, bootstrap aggregation (bagging)), and other model enhancement methods, etc.) one or more models produced in (e) based upon performance on validation sets obtained in (d) to form a composite model;
(g) combining (via boosting, committee machines etc,) a set of two or more models produced in (e or f) based upon performance on validation sets obtained in (d) to form a composite model; and
(h) running the model determined in either step (e), (f) or (g) using the required input data (the identity of the subset of input data itself was determined in step (c)) to predict the required target data.

13. The system of claim 12 wherein the analyses consider model biases and over training.

14. A method for predicting a characteristic of a chemical compound, the method comprising:
receiving as an input structure data for the compound; and
mapping the data to at least one chemical characteristic.

15. A predictive model of a chemical compound property produced according to the method of any of claims 1-3.

16. A computer readable medium containing a chemical compound characteristic model, the medium comprising:
a computer readable medium; and
a data structure on the medium that generates at least one characteristic for a compound from structure data and experimental or predictive data for the compound.

17. The medium of claim 16, wherein the characteristic is an ADME property.

18. The method of claim 17, wherein the ADME property is absorption.

19. The method of claim 16, wherein the characteristic is a toxic property.
